(19) United States
(12) Patent Application Publication
(10) Pub. No.: US 2022/0071807 A1
(43) Pub. Date: Mar. 10, 2022

(54) Flexible Wearable Foot Sling

(71) Applicant: Miriam Y. Salloum, Asheville, NC (US)

(72) Inventor: Miriam Y. Salloum, Asheville, NC (US)

(21) Appl. No.: 17/527,691

(22) Filed: Nov. 16, 2021

Related U.S. Application Data
(63) Continuation-in-part of application No. 16/287,932, filed on Feb. 27, 2019.
Application No. 16/287,932 is a continuation-in-part of application No. 14/881,812, filed on Oct. 13, 2015, now abandoned.
(60) Provisional application No. 62/063,139, filed on Oct. 13, 2014.

(51) Int. Cl. A61F 13/08; A43B 7/14; A61F 13/06

(52) U.S. Cl. CPC A61F 13/08; A61F 13/068; A61F 13/066; A43B 7/14

(57) ABSTRACT

A wearable foot sling for providing focal compression to portions of a wearer's foot, toes and leg, includes a sock member formed from an elastomeric fabric; a plantar surface portion having a thickened fabric pad; a plurality of tubes configured for engaged passage of the wearer's toes; a plurality of longitudinal fibers connecting the plurality of tubes with the thickened fabric pad; a plurality of toe sleeves configured for receipt of each toe of the wearer; a circumferential band forming a tension path beginning at a dorsum portion, wrapping laterally around at a lateral longitudinal arch portion and gradually widening as it runs medially at a medial longitudinal arch portion, and continuing upwards toward the dorsum portion to form a closed loop; the circumferential band continuing laterally and superior across an anterior ankle join portion, wrapping around a lower tibia portion and spiraling to the top of the sock member.

### RELATED APPLICATION

This application is a Continuation-in-Part application relating to and claiming the benefit of co-pending U.S. Nonprovisional patent application Ser. No. 16/287,932 filed Feb. 27, 2019, entitled “Flexible Wearable Foot Sling,” which claims priority to and incorporates entirely by reference U.S. Non-Provisional patent application Ser. No. 14/881,812 filed on Oct. 13, 2015, which claims priority to and incorporates entirely by reference U.S. Provisional Patent Application Ser. No. 62/063,139 filed on Oct. 13, 2014.

### FIELD OF THE INVENTION

This invention relates to medical devices and, more particularly, medical devices that support and assist individuals with musculoskeletal foot pain.

### BACKGROUND OF THE INVENTION

Human feet and ankles bear the brunt of every step we take. Women in particular wear uncomfortable, jarring yet fashionable shoes that can do damage to the structure of the foot over time. Athletes are also at risk of heightened foot problems from traumatic injury, joint, tendon, or ligament problems.

Both arthritis and foot pain are major public health problems. Approximately 24% of adults have foot ailments, and the prevalence increases with age. Surprisingly, this topic of musculoskeletal foot pain has received little attention in the rheumatology community.

Some of the most common foot problems experienced by individuals include, but are not limited to, foot and ankle trauma/fractures, ligament sprains/tendonitis, Achilles tendon problems, heel pain/plantar fasciitis, bunions, hammertoes or claw toes, ankle or foot arthritis and flat feet deformities, among other problems.

Despite the major focus of structure and alignment in arthritis, remarkably little work has focused on the foot and nonsurgical foot interventions that might affect lower extremity joint alignment, structure, and pain in rheumatic diseases. Emerging research suggests that there may be a significant role for foot orthotics and footwear in the treatment of rheumatoid arthritis, osteoarthritis of the hip, knee and foot and other commonly experienced conditions associated with musculoskeletal foot pain.

Because of these and other problems in the art, described herein, among other things, is a wearable foot sling for the relief of musculoskeletal foot pain comprised of materials possessing elastomeric properties that mimic the anatomical structures of the foot. In certain embodiments, the wearable foot sling described herein mimics the anatomical structures of the foot that may support the medial longitudinal arch and enhance the windlass mechanism during the late stance and push off phases of gait.

### SUMMARY OF THE INVENTION

In accordance with one form of this invention, there is provided a wearable foot sling for providing focal compression to portions of a wearer's foot, toes and leg, the foot sling including a sock member being formed from a first elastomeric material and surrounding a cavity sized for receipt of the foot, toes and leg of the wearer, the sock member further comprising a plurality of toe sleeves; a plantar surface portion having a thickened fabric pad forming a metatarsal bar, wherein the thickened fabric pad has a thickness greater than the first elastomeric material; a plurality of tubes each being sized and configured for engaged receipt and passage therethrough of the wearer's toes, the plurality of tubes being within the sock member and having openings aligned with the plurality of toe sleeves, such that when the foot sling is worn, each toe of the wearer is enclosed in a toe sleeve; an interdigit barrier separating each of the plurality of tubes; a plurality of longitudinal fibers configured to extend longitudinally from the plurality of tubes at a proximal phalanx portion to a heel portion; a circumferential band that is integrally formed with the sock member; the circumferential band forming a tension path that is configured to begin at a dorsum portion, configured to wrap laterally around at a lateral longitudinal arch portion and configured to gradually widen as it runs medially at a medial longitudinal arch portion, and configured to continue upwards toward the dorsum portion medially to connect with itself, thereby forming a closed loop; the circumferential band configured to continue laterally and superior across an anterior ankle joint portion, configured to wrap around a lower tibia portion; the circumferential band spiraling in a superior direction to a top portion of the sock member; and wherein the circumferential band is configured to apply tension along the tension path to the wearer's foot and leg.

In accordance with another form of this invention, there is provided a wearable foot sling for providing focal compression to portions of a wearer's foot, toes and leg, the foot sling including a sock member surrounding a cavity sized for receipt of the foot, toes and leg of the wearer, the sock member further comprising a plurality of toe sleeves; a plantar surface portion of the sock member having a thickened fabric pad forming a metatarsal bar; a plurality of tubes each being sized and configured for engaged receipt and passage therethrough of the wearer's toes, the plurality of tubes being within the sock member and having openings aligned with the toe sleeves, such that when the foot sling is worn, each toe of the wearer is enclosed in a toe sleeve; an interdigit barrier separating each of the plurality of tubes; a plurality of longitudinal fibers connecting the plurality of tubes with the thickened fabric pad, wherein the plurality of longitudinal fibers are configured to extend longitudinally from the plurality of tubes at a proximal phalanx portion to a heel portion; a circumferential band that is integrally formed with the sock member; the circumferential band forming a tension path configured to begin at a dorsum portion, configured to wrap laterally around at a lateral longitudinal arch portion and configured to gradually widen as it runs medially at a medial longitudinal arch portion, and configured to continue upwards toward the dorsum portion medially to connect with itself, thereby forming a closed loop; the circumferential band configured to wrap around a lower tibia portion; the circumferential band spiraling in a superior direction to a top portion of the sock member; and wherein the circumferential band is configured to apply tension along the tension path to the wearer's foot and leg.

In accordance with another form of this invention, there is provided a wearable foot sling for providing focal compression to portions of a wearer's foot, toes and leg, the foot sling including a sock member being formed from a first elastomeric material having a first tension force and surrounding a cavity sized for receipt of the foot, toes and leg of the wearer, the sock member further comprising a plurality of toe sleeves; a plantar surface portion of the sock member having a thickened fabric pad forming a metatarsal bar, wherein the thickened fabric pad has a thickness greater than the first elastomeric material; a plurality of tubes each being sized and configured for engaged receipt and passage therethrough of the wearer's toes, the plurality of tubes being within the sock member and having openings aligned with the toe sleeves, such that when the foot sling is worn, each toe of the wearer is enclosed in a toe sleeve; an interdigit barrier separating each of the plurality of tubes; a plurality of longitudinal fibers connecting the plurality of tubes with the thickened fabric pad; the plurality of longitudinal fibers configured to extend longitudinally from the plurality of tubes at a proximal phalanx portion to a heel portion; a circumferential band being formed from a second elastomeric material having a second tension force that is woven into the first elastomeric material of the sock member; wherein the second tension force is greater than the first tension force; the circumferential band forming a tension path configured to begin at a dorsum portion, configured to wrap laterally around at a lateral longitudinal arch portion and configured to gradually widen as it runs medially at a medial longitudinal arch portion, and configured to continue upwards toward the dorsum portion medially to connect with itself, thereby forming a closed loop; the circumferential band configured to continue laterally and superior across an anterior ankle joint portion, configured to wrap around a lower tibia portion; the circumferential band spiraling in a superior direction to a top portion of the sock member; and wherein the circumferential band is configured to apply tension along the tension path to the wearer's foot and leg.

### BRIEF DESCRIPTION OF THE DRAWINGS

For a fuller understanding of the nature of the present invention, reference should be made to the following detailed description, taken in conjunction with the accompanying drawings in which:

FIG. 1 is a perspective view of an embodiment of the flexible wearable foot sling;

FIG. 2 is a perspective view of an embodiment of the flexible wearable foot sling;

FIG. 3 is a perspective view of an embodiment of the flexible wearable foot sling;

FIG. 4 is a perspective view of an embodiment of the flexible wearable foot sling;

FIG. 5 is a perspective view of an embodiment of the flexible wearable foot sling;

FIG. 6 is a front elevational view of the flexible wearable foot sling in accordance with the embodiment illustrated in FIG. 5;

FIG. 7 is a rear elevational view of the flexible wearable foot sling in accordance with the embodiment illustrated in FIG. 5;

FIG. 8 is a right-side elevational view of the flexible wearable foot sling in accordance with the embodiment illustrated in FIG. 5;

FIG. 9 is a left-side elevational view of the flexible wearable foot sling in accordance with the embodiment illustrated in FIG. 5;

FIG. 10 is a top plan view of the flexible wearable foot sling in accordance with the embodiment illustrated in FIG. 5;

FIG. 11 is a bottom plan view of the flexible wearable foot sling in accordance with the embodiment illustrated in FIG. 5;

FIG. 12 is a perspective view of the flexible wearable foot sling in accordance with the embodiment illustrated in FIG. 5 being worn by a user;

FIG. 13 is a perspective view of an embodiment of the flexible wearable foot sling;

FIG. 14 is a front elevational view of the flexible wearable foot sling in accordance with the embodiment illustrated in FIG. 13;

FIG. 15 is a rear elevational view of the flexible wearable foot sling in accordance with the embodiment illustrated in FIG. 13;

FIG. 16 is a right-side elevational view of the flexible wearable foot sling in accordance with the embodiment illustrated in FIG. 13;

FIG. 17 is a left-side elevational of the flexible wearable foot sling in accordance with the embodiment illustrated in FIG. 13;

FIG. 18 is a top plan view of the flexible wearable foot sling in accordance with the embodiment illustrated in FIG. 13;

FIG. 19 is a bottom plan view of the flexible wearable foot sling in accordance with the embodiment illustrated in FIG. 13;

FIG. 20 is a perspective view of the flexible wearable foot sling in accordance with the embodiment illustrated in FIG. 13 being worn by the user; and

FIG. 21 illustrates a cutaway view along line 21 of FIG. 14.

Like reference numerals refer to like parts throughout the several views of the drawings.

### DETAILED DESCRIPTION OF THE PREFERRED EMBODIMENTS

In general, the wearable foot sling 101 disclosed herein is designed to address relief of musculoskeletal foot pain and is designed for comfort and compatibility with most shoes worn, including low profile dress and sport shoes.

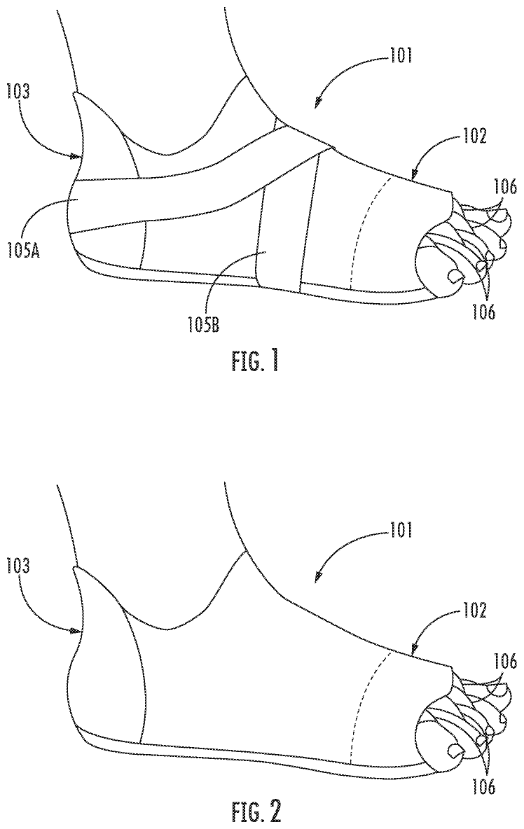

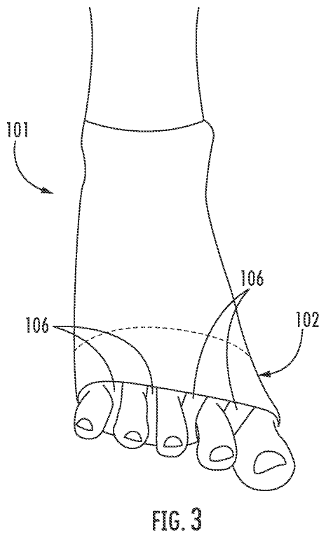

In certain embodiments, the wearable foot sling 101 will be comprised of a conforming material with elastomeric properties known to those of ordinary skill in the art. Contemplated materials include, but are not necessarily limited to, neoprene, polychloroprenes and other known synthetic rubbers with elastomeric properties. In one embodiment, as depicted in FIGS. 1-2, the wearable foot sling 101 is comprised of a tight-fitting sling 101, the sling 101 having a front end 102 which is comprised of webbing 106 that fits over the interdigit spaces of toes 1-5 and a back end 103 which is secured over the posterior heel. Once the sling 101 is positioned in place on a user's foot, the sling 101 functions to restrain the longitudinal lengthening of soft tissues between a user's toes and heel, effectively shortening the fibers of the plantar fascia.

In certain alternative embodiments, the sling 101 will include bands of elastomeric properties aligned in parallel with the metatarsals that will augment the shortening effect applied by the sling 101. In general, any material known to those of ordinary skill in the art with elastomeric properties is contemplated as a possible band aligned in parallel with the metatarsals.

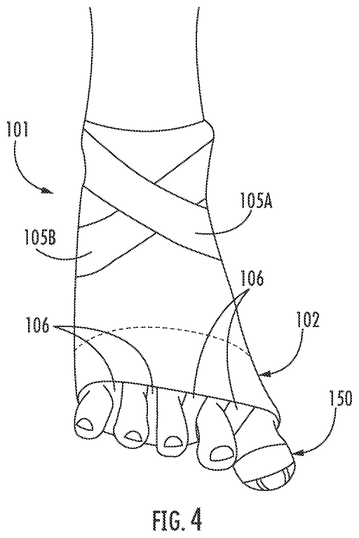

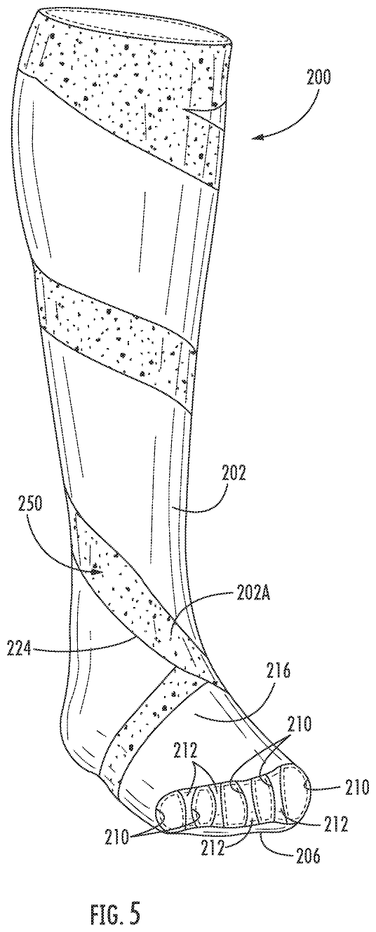

In other embodiments of the sling 101, there will be a pair of diagonally oriented supports 105A, 105B that cross under the plantar midfoot to further support the medial longitudinal arch. In certain embodiments, as demonstrated in FIGS. 1 and 4, the diagonally oriented supports 105A, 105B will also cross diagonally on the top of the foot and sling 101, although this orientation is not determinative. As depicted in FIGS. 1 and 4, the medial longitudinal diagonally oriented supports 105A, 105B will likely be secured over the dorsum of the foot by fastening means known to those of ordinary skill in the art including, but not limited to, hook and loop fastener, clips, hooks, ties, fasteners, knots, and loops. In one embodiment, the diagonally oriented supports 105A, 105B are made from an elastomeric material.

In certain additional embodiments, an additional element of the sling 101 is a smaller sling system around the first phalanx of the great toe, the phalanx sling 150. As demonstrated in FIG. 4, the phalanx sling 150 is comprised of a single support that attaches to the bottom of the sling 101 at the base of the great toe, wraps around the front of the great toe and then crosses back over itself to attach to the bottom of the sling 101 at the base of the great toe on the opposite side. Accordingly, the phalanx sling 150 wraps around the front of the toe and forms a cross and attaches to the bottom of the sling near the great toe. In general, the function of the phalanx sling 150 is to improve plantar flexion during loading. It should be understood that this phalanx sling 150 may be optional, and could be reversed (i.e., the phalanx sling may be wrapped around the great toe in the opposite manner than demonstrated in FIG. 4) to enhance dorsiflexion.

In sum, the orientation of the sling 101 provided herein creates a multi-point harness which shortens and restricts the motion of the plantar structures of the foot in a triplanar manner, thereby decreasing abnormal tension in muscle, fascia, and neural pathways. As such, this device is similar in purpose to both the molded and non-molded foot orthoses of the prior art which are utilized for decreasing foot pain.

However, the sling 101 described herein has numerous advantages over these designs of the prior art. First, as demonstrated in FIGS. 1-4, the sling 101 has a lower profile, fitting snugly with the foot. This lowered profile allows compatibility between the sling 101 and most shoe designs. Further, unlike the designs of the prior art, because of its low profile a user can use the sling generally without removing the original insoles of the shoes. Second, the sling offers the possibility for increased customer compliance and more frequent use than the currently utilized methodologies due to decreased sense of difficulty with use (compared to prior art technologies) and increased sense of comfort. Third, due to its simple design, the sling 101 is more reasonably priced than the custom foot orthoses of the prior art. In addition, the currently utilized over-the-counter and custom orthoses frequently have to be accommodated by either the purchase of a separate pair of shoes to account for increased depth and width of the orthoses materials, also adding to the cost. As noted previously, this is not necessary with the disclosed sling 101. Finally, another issue with current foot orthoses utilized in the art is the incompatibility of a person's foot function and the density and contours of the materials comprising the orthoses. Pain, discomfort and blisters are frequent results of such issues with errors in fit and material density. Non-compliance by patients due to discomfort and cost caused by the orthoses of the prior art is a frequently cited drawback in some studies on foot pain and orthoses.

In certain embodiments, the sling 101 disclosed herein will be utilized as follows. In a first step, the front end 102 of the sling, which is comprised of webbing 106 that fits over the interdigit spaces of toes 1-5, is placed in the interdigit spaces of a user's toes. Generally this first step is performed while the user is in a non-weight bearing position (e.g., sitting). In a second step, the back end 103 of the sling 101 is secured over the user's posterior heel, bringing the sling into a snug, taut and comfortable position on the bottom of a user's foot. In a third step, the user secures the diagonally oriented supports 105A, 105B (proximal/lateral to distal/medial and distal/lateral to proximal/medial midfoot) to create a hammock for the navicular. In a fourth step, in the embodiments of the sling 101 with a phalanx sling 150, the phalanx sling 150 is positioned into place around the great toe. In these embodiments, this phalanx sling assists the proximal phalanx of the first toe in plantar flexion, but does not necessarily restrict overall mobility. As noted previously, this phalanx sling 150 may be reversed to the dorsum or removed based on the comfort to the customer.

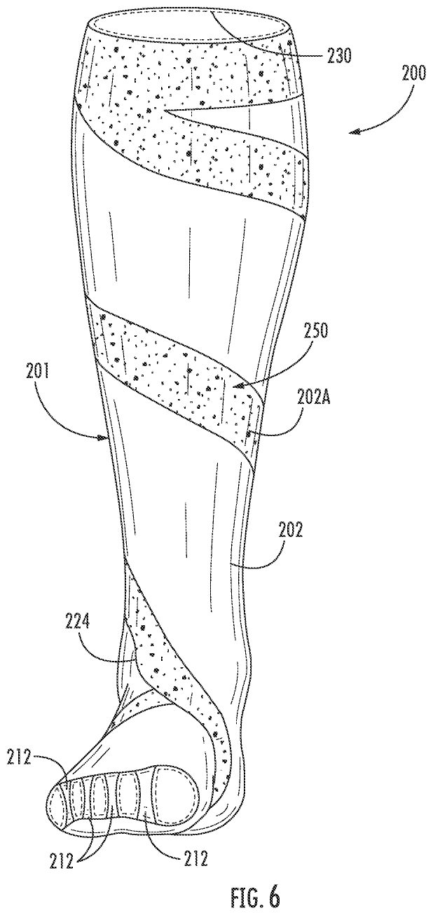

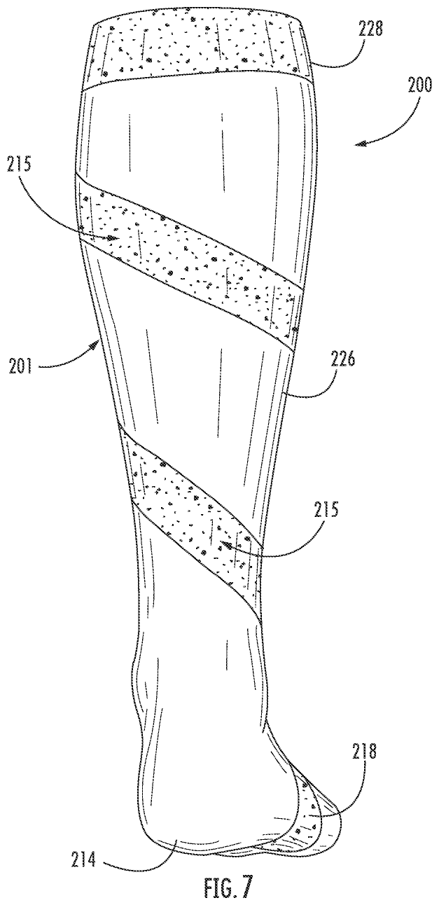

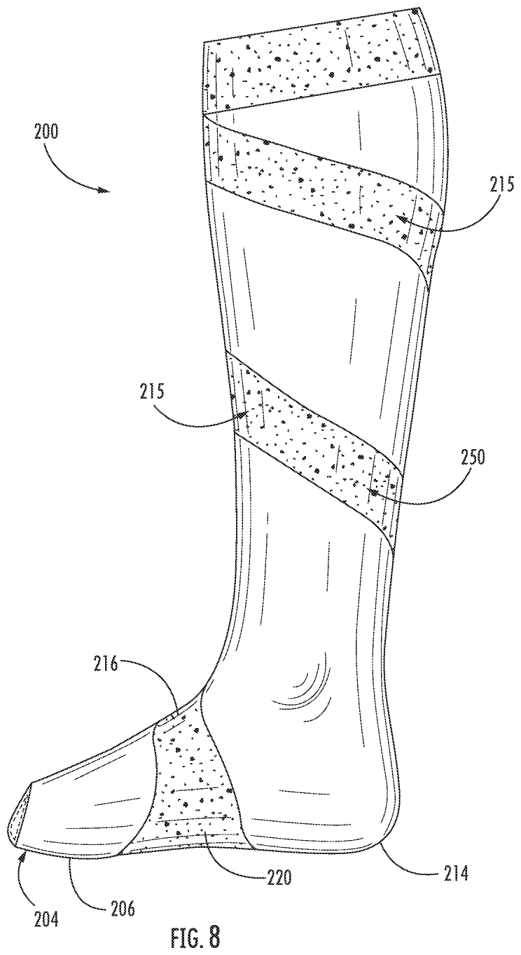

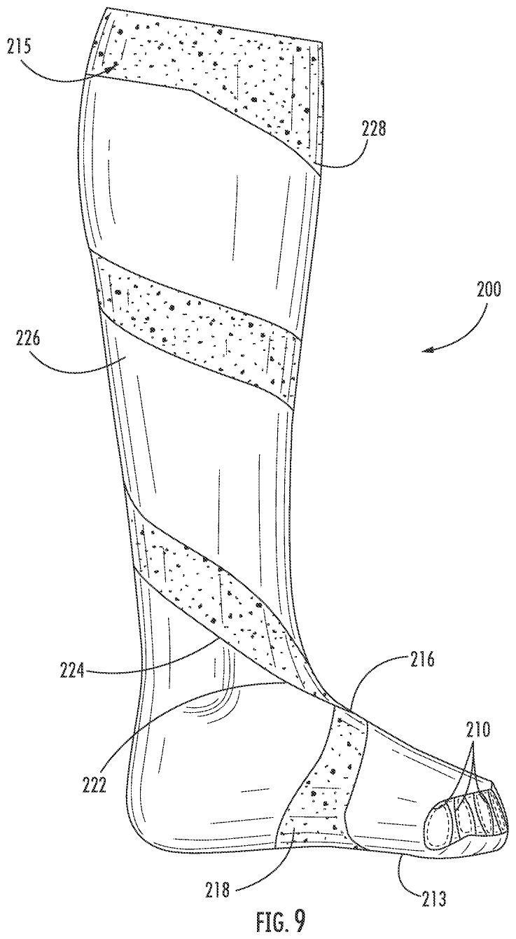

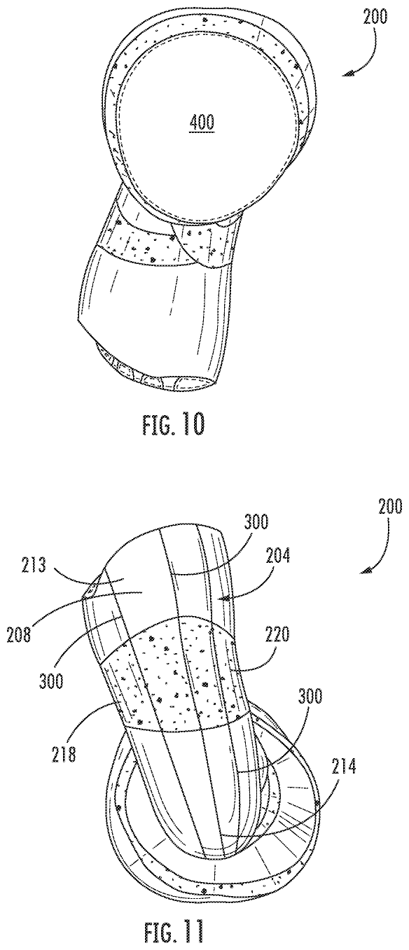

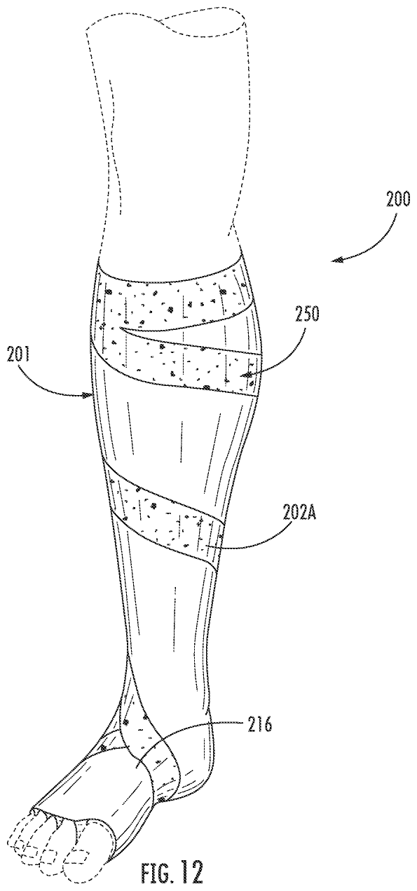

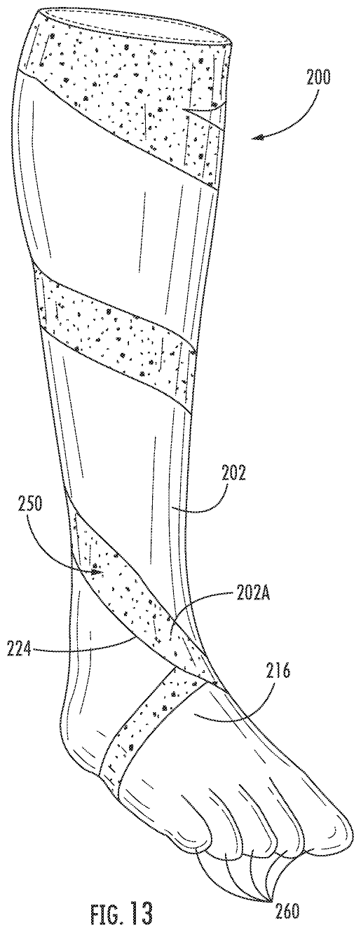

Referring now to FIGS. 5-12, another embodiment of the wearable foot sling 200 is shown and includes a sock member 201 that is primarily formed from a first elastomeric fabric 202 surrounding a cavity 400 that is sized and configured for receipt of a wearer's foot, toes, and a portion of the wearer's leg.

At the plantar surface portion 204 of the sling 200 there is a thicker weave forming a thickened fabric pad 206 having a thickness greater than the elastomeric fabric 202. The thickened fabric pad 206 is positioned proximal the metatarsal heads of the wearer's foot and is sized and configured for serving as a metatarsal bar 208 to create an unloading effect on the metatarsal heads. The thickened fabric pad 206 connects with a plurality of longitudinal fibers 300 of the plurality of tubes 210, separating the wearer's toes, thereby addressing issues of sesamoiditis, metatarsalgia, and plantar fasciitis.

The tubes 210 are formed of thickened fabric and are sized and configured for engaged receipt and passage therethrough of the wearer's toes, which are separated individually by interdigit barriers 212, thereby leaving the digits of the wearer's foot either exposed. In some embodiments of the sling 200 a light weave may be included to cover the wearer's toes (not shown). The main function of the interdigit barriers 212 between the toes (between the base of the proximal phalanx) is to increase the space between the metatarsal heads in a longitudinal manner and to decrease impingement of the interdigital nerves (such as with Morton's neuroma and metatarsalgia). In one embodiment, the longitudinal fibers 300 extend longitudinally between the plurality of tubes 210 at a proximal phalanx portion 213 and the heel portion 214 will further unload the plantar fascia and intrinsic foot muscles by shortening in the sagittal. This will be an additional plane by which the plantar soft tissue is unloaded, which will assist the circumferential band 250 that primarily serves to unload in the frontal plane.

The circumferential band 250 is integrally formed with the sock member 201 as part of the sling 200 formed from a second elastomeric material 202A provides increased tension along a tension path 215 on the wearer relative to the tension supplied by the elastomeric fabric 202. In one embodiment, the circumferential band 250 is woven into the elastomeric fabric 202 of the sling 200. The circumferential band 250 starts on the dorsum portion 216 of the sling 200, wraps laterally around at the lateral longitudinal arch portion 218 and gradually widens as it runs medially at the medial longitudinal arch portion 220, and continues back up toward the dorsum portion 216 medially to partially connect to the starting point, thereby creating a closed loop. From there, the band 250 partially continues laterally and superior to cross the anterior ankle joint portion 222, wrapping around the distal lateral aspect of the lower tibia portion 224, located just above the lateral malleolus of the wearer, and continues to wrap around diagonally from the lateral to medial aspect of the lower tibia portion 224 as it spirals in a superior direction to the top 230 of the sling 201. This upward spiraling weave will effectively cinch the soft tissue structure of the wearer at the posterior medial compartment portion 226, adjacent the soleus, flexor digitorum longus and posterior tibialis muscles of the wearer, to the tibial periosteum portion 228. This diagonal compression of these muscles toward the tibia will address bony traction injuries like medial tibial stress syndrome and foot/ankles pain of posterior tibialis tendinopathy. Focal and specific compression of these muscles mimic successful athletic taping techniques demonstrated in the research.

Generally, the sling 200 uses a focal fabric weave with high levels of compression in diagonal patterns, therefore mimicking the triplanar motion of the foot and lower leg versus just straight planes of compression typically seen with other designs. This design makes the strapping system more biomechanically effective for treating foot/ankle/lower leg pain. Moreover, the strapping system of this higher compression weave is made more focal and concentrated with unloading the specific structures of the foot/lower leg mentioned above due to it being surrounded by a less compressive weave. This makes a true “strap” system with defined areas of compression and control.

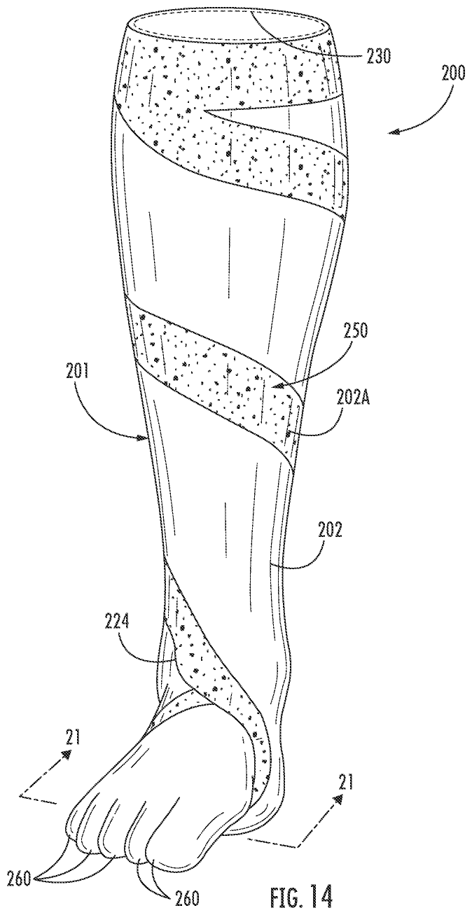

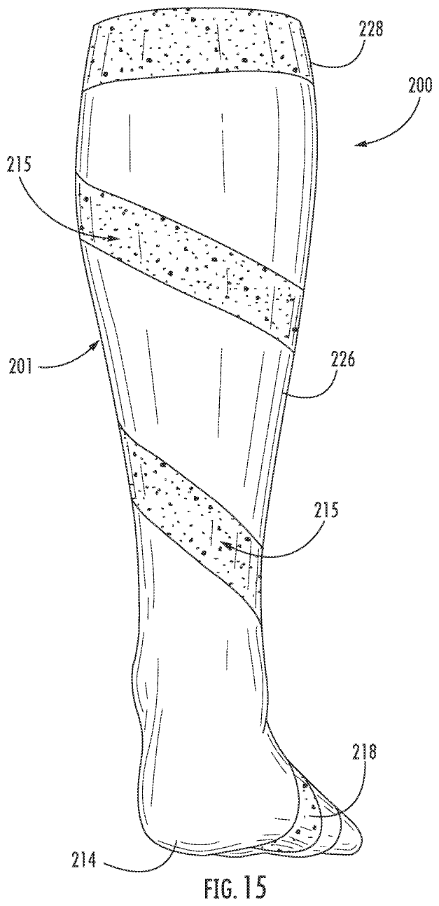

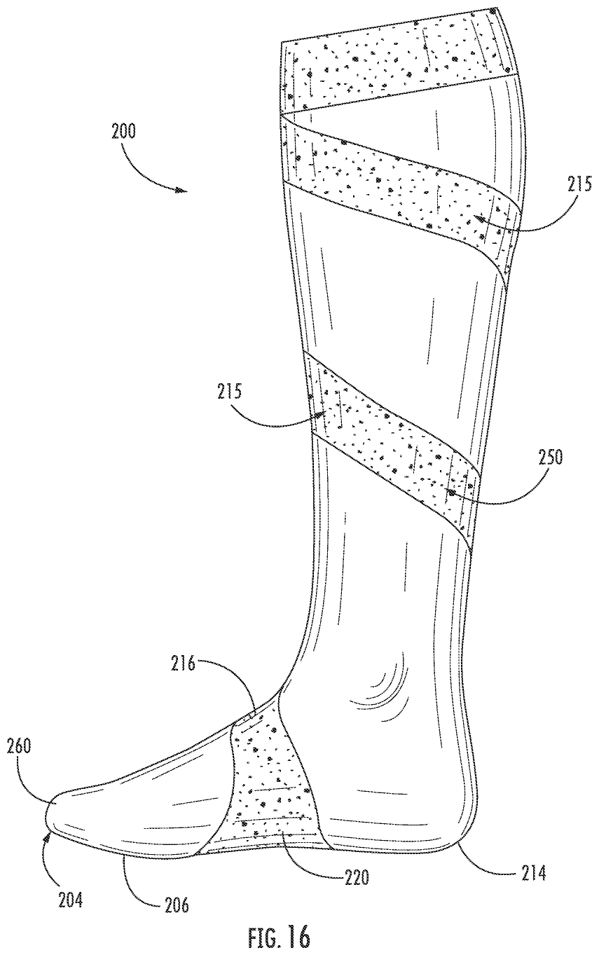

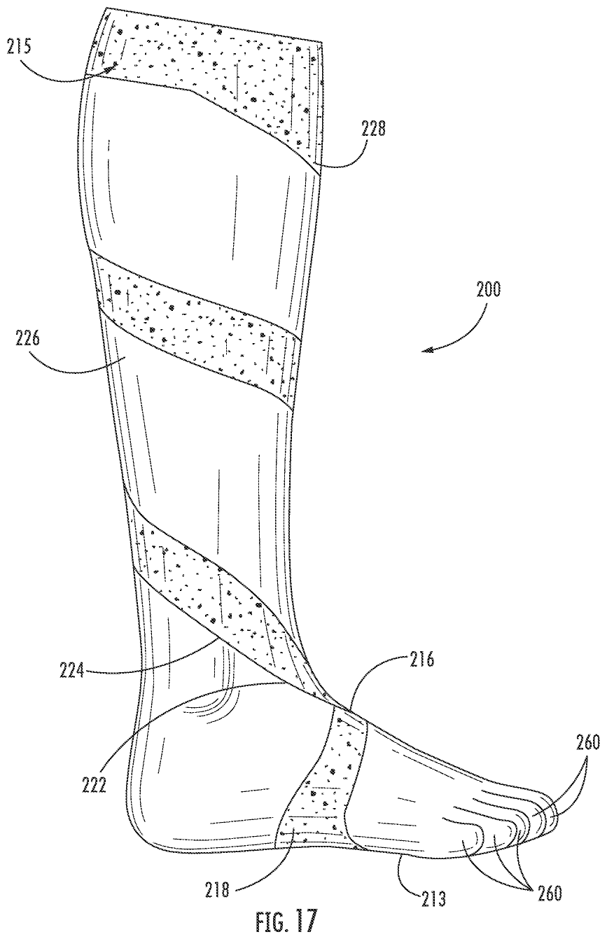

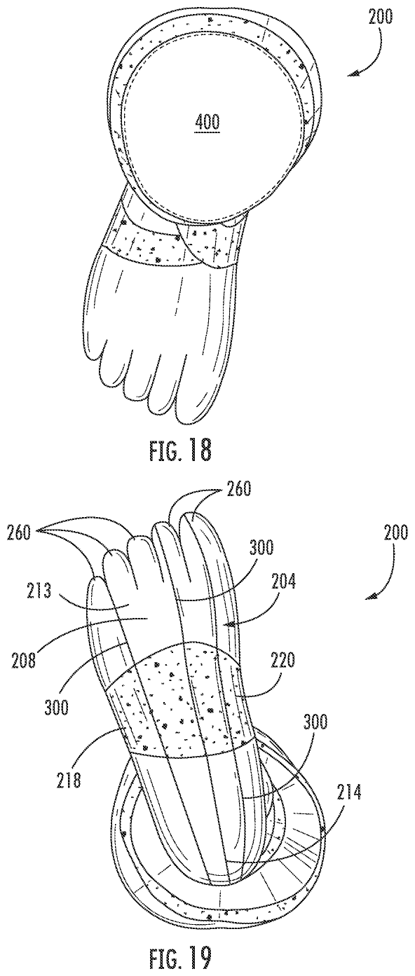

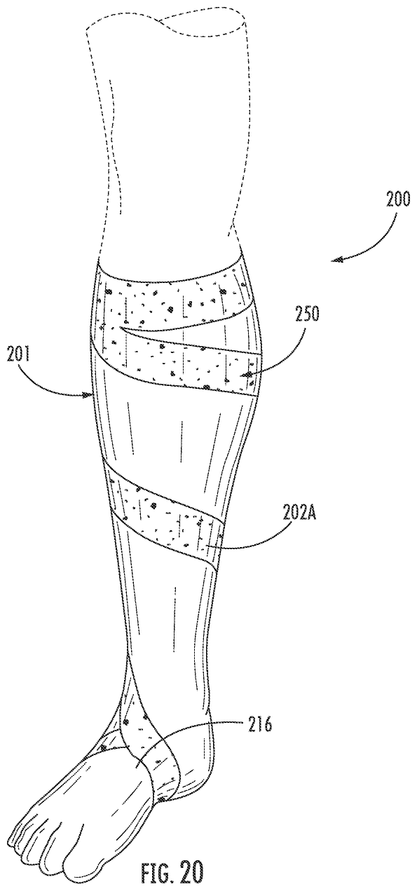

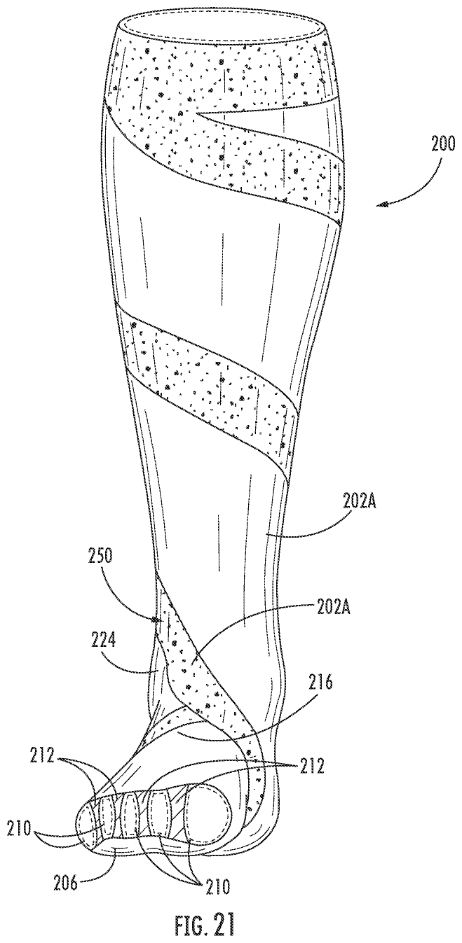

Referring now to FIGS. 13-21, an embodiment of the sling 200 is shown in which a plurality of toe sleeves 260 are included, in which each of a user's individual toes is covered. The toe sleeves may be composed of a light weave fabric, including the fabric that is used elsewhere in the sling 200, such as the elastomeric fabric 202 or other contemplated materials to include neoprene, polychloroprenes, or other known synthetic fabrics or materials with elastomeric properties.

As with other embodiments, the embodiment shown in FIGS. 13-21 includes a sock member 201 that is primarily formed from a first elastomeric fabric 202 surrounding a cavity 400 that is sized and configured for receipt of a wearer's foot, toes, and a portion of the wearer's leg.

At the plantar surface portion 204 of the sling 200 there is a thicker weave forming a thickened fabric pad 206 having a thickness greater than the elastomeric fabric 202. The thickened fabric pad 206 is positioned proximal the metatarsal heads of the wearer's foot and is sized and configured for serving as a metatarsal bar 208 to create an unloading effect on the metatarsal heads. The thickened fabric pad 206 connects with a plurality of longitudinal fibers 300 of the plurality of tubes 210, separating the wearer's toes, thereby addressing issues of sesamoiditis, metatarsalgia, and plantar fasciitis.

The tubes 210 are formed of thickened fabric and are sized and configured for engaged receipt and passage therethrough of the wearer's toes, which are separated individually by interdigit barriers 212, and the individual digits of the wearer's foot are enclosed by toe sleeves 260. In some embodiments of the sling 200 a light weave may be included in the toe sleeves 260 to cover the wearer's toes. As with other embodiments, the presence of toe sleeves 260 does not preclude the presence of interdigit barriers 212 between the toes (between the base of the proximal phalanx), which may serve to increase the space between the metatarsal heads in a longitudinal manner and to decrease impingement of the interdigital nerves (such as with Morton's neuroma and metatarsalgia). In one embodiment, the longitudinal fibers 300 extend longitudinally between the plurality of tubes 210 at a proximal phalanx portion 213 and the heel portion 214 will further unload the plantar fascia and intrinsic foot muscles by shortening in the sagittal. This will be an additional plane by which the plantar soft tissue is unloaded, which will assist the circumferential band 250 that primarily serves to unload in the frontal plane.

The circumferential band 250 is integrally formed with the sock member 201 as part of the sling 200 formed from a second elastomeric material 202A and provides increased tension along a tension path 215 on the wearer relative to the tension supplied by the elastomeric fabric 202. In one embodiment, the circumferential band 250 is woven into the elastomeric fabric 202 of the sling 200. The circumferential band 250 starts on the dorsum portion 216 of the sling 200, wraps laterally around at the lateral longitudinal arch portion 218 and gradually widens as it runs medially at the medial longitudinal arch portion 220, and continues back up toward the dorsum portion 216 medially to partially connect to the starting point, thereby creating a closed loop. From there, the band 250 partially continues laterally and superior to cross the anterior ankle joint portion 222, wrapping around the distal lateral aspect of the lower tibia portion 224, located just above the lateral malleolus of the wearer, and continues to wrap around diagonally from the lateral to medial aspect of the lower tibia portion 224 as it spirals in a superior direction to the top 230 of the sling 201. This upward spiraling weave will effectively cinch the soft tissue structure of the wearer at the posterior medial compartment portion 226, adjacent the soleus, flexor digitorum longus and posterior tibialis muscles of the wearer, to the tibial periosteum portion 228. This diagonal compression of these muscles toward the tibia will address bony traction injuries like medial tibial stress syndrome and foot/ankles pain of posterior tibialis tendinopathy. Focal and specific compression of these muscles mimic successful athletic taping techniques demonstrated in the research.

Generally, the sling 200 uses a focal fabric weave with high levels of compression in diagonal patterns, therefore mimicking the triplanar motion of the foot and lower leg versus just straight planes of compression typically seen with other designs. This design makes the strapping system more biomechanically effective for treating foot/ankle/lower leg pain. Moreover, the strapping system of this higher compression weave is made more focal and concentrated with unloading the specific structures of the foot/lower leg mentioned above due to it being surrounded by a less compressive weave. This makes a true “strap” system with defined areas of compression and control.

While the invention has been disclosed in connection with certain preferred embodiments, the elements, connections, and dimensions of the preferred embodiments should not be understood as limitations on all embodiments. Modifications and variations of the described embodiments may be made without departing from the spirit and scope of the invention, and other embodiments should be understood to be encompassed in the present disclosure as would be understood by those of ordinary skill in the art.

What is claimed is:

1. A wearable foot sling for providing focal compression to portions of a wearer's foot, toes and leg, the foot sling comprising:
a sock member being formed from a first elastomeric material and surrounding a cavity sized for receipt of the foot, toes and leg of the wearer, the sock member further comprising a plurality of toe sleeves; a plantar surface of the sock member having a thickened fabric pad forming a metatarsal bar, wherein the thickened fabric pad has a thickness greater than the first elastomeric material; a plurality of tubes each being sized and configured for engaged receipt and passage therethrough of the wearer's toes, the plurality of tubes being within the sock member and having openings aligned with the plurality of toe sleeves, such that when the foot sling is worn, each toe of the wearer is enclosed in a toe sleeve; an interdigit barrier separating each of the plurality of tubes; a plurality of longitudinal fibers connecting the plurality of tubes with the thickened fabric pad, wherein the plurality of longitudinal fibers are configured to extend longitudinally from the plurality of tubes at a proximal phalanx portion to a heel portion;
a circumferential band that is integrally formed with the sock member; the circumferential band forming a tension path that is configured to begin at a dorsum portion, configured to wrap laterally around at a lateral longitudinal arch portion and configured to gradually widen as it runs medially at a medial longitudinal arch portion, and configured to continue upwards toward the dorsum portion medially to connect with itself, thereby forming a closed loop; the circumferential band configured to continue laterally and superior across an anterior ankle joint portion, configured to wrap around a lower tibia portion; the circumferential band spiraling in a superior direction to a top portion of the sock member; and
wherein the circumferential band is configured to apply tension along the tension path to the wearer's foot and leg.

2. The wearable foot sling of claim 1 wherein the circumferential band is woven into the first elastomeric material of the sock member.

3. The wearable foot sling as recited in claim 1 wherein the first elastomeric material of the sock member has a first tension force; the circumferential band is formed from a second elastomeric material having a second tension force; and wherein the second tension force is greater than the first tension force.

4. A wearable foot sling for providing compression to portions of a wearer's foot, toes and leg, the foot sling comprising:
a sock member surrounding a cavity sized for receipt of the foot, toes and leg of the wearer, the sock member further comprising a plurality of toe sleeves; a plantar surface portion of the sock member having a thickened fabric pad forming a metatarsal bar; a plurality of tubes each being sized and configured for engaged receipt and passage therethrough of the wearer's toes, the plurality of tubes being within the sock member and having openings aligned with the toe sleeves, such that when the foot sling is worn, each toe of the wearer is enclosed in a toe sleeve; an interdigit barrier separating each of the plurality of tubes; a plurality of longitudinal fibers connecting the plurality of tubes with the thickened fabric pad, wherein the plurality of longitudinal fibers are configured to extend longitudinally from the plurality of tubes at a proximal phalanx portion to a heel portion;
a circumferential band that is integrally formed with the sock member; the circumferential band forming a tension path configured to begin at a dorsum portion, configured to wrap laterally around at a lateral longitudinal arch portion and configured to gradually widen as it runs medially at a medial longitudinal arch portion, and configured to continue upwards toward the dorsum portion medially to connect with itself, thereby forming a closed loop; the circumferential band configured to wrap around a lower tibia portion; the circumferential band spiraling in a superior direction to a top portion of the sock member; and
wherein the circumferential band is configured to apply tension along the tension path to the wearer's foot and leg.

5. The wearable foot sling of claim 4 wherein the sock member is formed from a first elastomeric material.

6. The wearable sling of claim 4 wherein the circumferential band is woven into the first elastomeric material of the sock member.

7. The wearable foot sling as recited in claim 5 wherein the first elastomeric material of the sock member has a first tension force; the circumferential band is formed from a second elastomeric material having a second tension force; and wherein the second tension force is greater than the first tension force.

8. A wearable foot sling for providing focal compression to portions of a wearer's foot, toes and leg, the foot sling comprising:
a sock member being formed from a first elastomeric material having a first tension force and surrounding a cavity sized for receipt of the foot, toes and leg of the wearer, the sock member further comprising a plurality of toe sleeves; a plantar surface portion of the sock member having a thickened fabric pad forming a metatarsal bar, wherein the thickened fabric pad has a thickness greater than the first elastomeric material; a plurality of tubes each being sized and configured for engaged receipt and passage therethrough of the wearer's toes, the plurality of tubes being within the sock member and having openings aligned with the toe sleeves, such that when the foot sling is worn, each toe of the wearer is enclosed in a toe sleeve; an interdigit barrier separating each of the plurality of tubes; a plurality of longitudinal fibers connecting the plurality of tubes with the thickened fabric pad; the plurality of longitudinal fibers configured to extend longitudinally from the plurality of tubes at a proximal phalanx portion to a heel portion;
a circumferential band being formed from a second elastomeric material having a second tension force that is woven into the first elastomeric material of the sock member; wherein the second tension force is greater than the first tension force; the circumferential band forming a tension path configured to begin at a dorsum portion, configured to wrap laterally around at a lateral longitudinal arch portion and configured to gradually widen as it runs medially at a medial longitudinal arch portion, and configured to continue upwards toward the dorsum portion medially to connect with itself, thereby forming a closed loop; the circumferential band configured to continue laterally and superior across an anterior ankle joint portion, configured to wrap around a lower tibia portion; the circumferential band spiraling in a superior direction to a top portion of the sock member; and
wherein the circumferential band is configured to apply tension along the tension path to the wearer's foot and leg.
